(19) United States
(12) Patent Application Publication
(10) Pub. No.: US 2007/0192133 A1
(43) Pub. Date: Aug. 16, 2007

(54) Patient record system

(76) Inventor: David W. Morgan, Birmingham (GB)

Correspondence Address: KENYON & KENYON LLP, ONE BROADWAY, NEW YORK, NY, 10004, US

(21) Appl. No.: 11/509,442

(22) Filed: Aug. 23, 2006

Related U.S. Application Data
(63) Continuation of application No. PCT/GB05/00670, filed on Feb. 23, 2005.

(30) Foreign Application Priority Data
Feb. 23, 2004 (GB) 0403947.5

(51) Int. Cl. G06Q 10/00; A61B 5/00

(52) U.S. Cl. 705/2; 600/300

(57) ABSTRACT

A patient record system operable to store a patient record corresponding to a patient and having patient information, the patient record system being operable, as part of a medical process, to; receive a request for patient information from an input/output device, retrieve patient information from the patient record, transmit the patient information to the input/output device, receive verification information from the input/output device, and identify the patient as being ready for a next stage of the medical process in accordance with the verification information.

### CROSS-REFERENCE TO RELATED APPLICATIONS

This application is a continuation of international PCT application Serial No. PCT/GB05/000670 filed Feb. 23, 2005 and published as WO2005/083614 in English on Sep. 9, 2005, which claims priority under 35 U.S.C. §119 to Great Britain application Serial No. GB0403947.5 filed Feb. 23, 2004.

### BACKGROUND OF THE INVENTION

The invention relates to a patient records system, particularly for improving the safety of patients during a medical process, and in particular reducing the risk of carrying out surgery at the wrong site on a patient.

The planning of a medical process and more particularly a perioperative process, i. e. a process before, during and after surgery, for a patient is the responsibility of several, loosely-coupled systems in a hospital, for example, systems carried out by admissions secretaries, ward nurses, surgeons and anaesthetists. This involves the manual updating of patient information, for example details of the patient, e. g. identity, and details of the type of surgery and the site of the surgery to be carried out. This can lead to a number of problems, for example wrong side/site surgery (WSS), which is a major cause of morbidity and litigation in the UK National Health Service (NHS). A common cause of WSS is last minute written changes made to an operating list which causes confusion and mistakes. On a given operating list, identification of the patient, and of the type and site of the surgery to be carried out, can often be manually changed several times. Patients undergoing surgery may be incorrectly identified and/or undergo the wrong surgery or surgery at the wrong site.

### SUMMARY OF THE INVENTION

According to a first aspect of the invention, provided is a patient record system operable to store a patient record corresponding to a patient and comprising patient information, the patient record system being operable, as part of a medical process, to receive a request for patient information from an input/output device, retrieve patient information from the patient record, transmit the patient information to the input/output device, receive verification information from the input/output device, and identify the patient as being ready for a next stage of the medical process in accordance with the verification information.

The patient record may include a tag identifier corresponding to a wireless tag associated with the patient, the patient record system being operable, in response to the request for patient information from an input/output device to obtain location information for the wireless tag corresponding to the tag identifier, transmit the location information to the input/output device.

The patient record system may be operable to transmit the location information with the patient information.

The input/output device may comprise a portable device such as a personal digital assistant.

The patient record system may be operable to receive location information for a wireless tag having a tag identifier indicating the presence of the wireless tag at a specific location, identify the patient record including the tag identifier of the wireless tag, and verify the patient is expected at that location.

The patient record system may be operable to generate an alarm if the location is incorrect.

The patient record system may be operable to supply patient information from the patient record to an input/output device at the location if the location is correct.

The input/output device may comprise an input/output device associated with the location.

The medical process may be a perioperative process.

The patient record system may be operable, as part of a perioperative process, to receive verification information from the input/output device, and identify the patient as being ready for a next stage of the perioperative process in accordance with the verification information.

The step of receiving verification information comprises generating a checklist, receiving an input in response to the check list, and generating verification information in accordance with the input.

The patient record system may be operable to obtain location information for the wireless tag corresponding to the tag identifier, and transmit the location information to the input/output device.

The patient information may comprise status information and wherein the status information is modified in response to the verification information.

The location information may be displayed as a plan of a building with the patient location indicated on the plan.

The patient information may comprise status information and the patient location may be color coded in accordance with the status information.

The patient record information may comprise an image of the patient and the step of transmitting the patient information includes transmitting the image.

The step of receiving a request for patient information may comprise displaying a list of patients and receiving a indication corresponding to one of the list of patients.

According to a second aspect of the invention there is provided a medical process for a patient, comprising creating a computerized record for the patient including a photograph of the patient, and at at least one subsequent stage of the medical process, accessing the computerized patient record and checking that the patient undergoing the perioperative process matches the patient in the photograph of the record.

The stage of the medical process comprises a nurse's assessment of the fitness of the patient for surgery, or a surgeon's assessment of the fitness of the patient for surgery, or an anaesthetist's assessment of the fitness of the patient for surgery, or administration of an anaesthetic to the patient, or carrying out surgery on the patient.

Throughout the medical process, the identity of the patient can be continually checked using the computerized photograph and the location information obtained from the wireless tag, thus minimizing the chance that any part of the perioperative process, particularly the administration of the anaesthetic and the surgery, is carried out on the wrong patient.

The computerized patient record may be stored in a patient database. The computerized patient record may be capable of amendment by one or more members of staff, e. g. admission secretaries, nurses, anaesthetists or surgeons. The amendments may be stored in the patient record in the patient database. Thus an up-to-date patient record can easily be maintained, and can be easily accessed by the various members of staff. The computerized patient record may be protected such that it can only be accessed by members of staff who are authorized to do so. For example, details of the members of staff who are authorized to access the computerized patient record may be held in a staff database. Verification of a member if staff wishing to access the computerized patient record may be made prior to access, by comparing details entered by the member of staff to those held on the staff database. Using a computerized patient record for the patient, may help to expedite the perioperative process for the patient.

The computerized patient record may include one or more of: name, identification number, gender, and age of the patient, date of admission of the patient, ward number/name/reference code to which the patient is being admitted, name/identification number of the members of staff responsible for the care of the patient, identification number/description, date and start time of surgery to be carried out on the patient, surgery site of the patient, type/reference code of the anaesthetic to be used on the patient, code/identification number of operating theatre where the surgery is to take place, editable notes on the patient, status information on the current state of the patient, known allergies or infections, etc.

The computerized patient record may include details of the surgery site of the patient, and the record may be used to check these details prior to carrying out the surgery.

Using a computerized patient record which contains the details of the surgery site, allows the site of the surgery to be amended as required, e. g. by the surgeon when assessing the patient for surgery. Any amendment may be stored in the patient record in the patient database, by overwriting any existing surgery site details. Up-to-date surgery site details may therefore be readily obtained by the surgeon and other members of staff. As amended surgery site details overwrite any other such details, the up-to-date details may be easily read by the surgeon. Immediately prior to carrying out the surgery, the surgeon and the anaesthetist and a scrub nurse can use the patient record to access the up-to-date surgery site details and confirm the patient's identity. In this perioperative process, the risk of WSS is thereby reduced.

According to a third aspect of the invention there is provided a system for implementing the medical process according to the first aspect of the invention, comprising a server, an input/output device for use with the server for creating a computerized patient record, and an input/output device for use with the server for accessing the computerized patient record.

The input/output devices may comprise any of: a computer, a graphics tablet, a Personal Digital Assistant (PDA).

According to a fourth aspect of the invention there is provided a method of supplying patient information comprising storing a patient record comprising patient information, receiving a request for patient information from an input/output device retrieving patient information from the patient record, transmitting the patient information to the input/output device, receiving verification information from the input/output device, and identifying the patient as being ready for a next stage of a medical process in accordance with the verification information.

The method may comprise associating a wireless tag having a tag identifier with the patient and, storing the tag identifier in the patient record, and in response to the request, obtaining location information for the wireless tag corresponding to the tag identifier, and transmit the location information to the input/output device.

The computerized patient record may be displayed on an input/output device which is located in proximity to the position of the tag. The display of the computerized patient record may be used to check that the patient undergoing the perioperative process matches the patient in the record. The display of the computerized patient record may comprise a photograph, particularly of the patient's face of the patient and this may be used to check that the patient undergoing the perioperative process matches the patient in the record.

In this way, when the patient is moved to the anaesthetics room, for example, the tag assigned to the patient may be used to determine that the patient is in the anaesthetics room, and the tag identification may be used to call up and display the record of the patient on an input/output device in the room. The anaesthetist may therefore look at the displayed record, and check that the patient which has arrived in the room is the same as the patient whose record is being displayed using the photograph contained in the displayed record.

The tag may comprise a radio frequency identification (RFID) tag. The position of the RFID tag may be determined using one or more radio frequency signal sensors and a wireless positioning engine.

According to a fifth aspect of the invention there is provided a system for implementing the perioperative process according to the third aspect of the invention, comprising a tag, tag position detector, a server, an input/output device for use with the server for creating a computerized patient record, an input/output device for use with the server for accessing the computerized patient record, and an input/output device for use with the server for displaying the computerized patient record.

### BRIEF DESCRIPTION OF THE DRAWINGS

Embodiments of the invention will now be described by way of example only, with reference to the accompanying drawings, in which:

FIG. 1 is a schematic representation of a system of hardware used to carry out the perioperative process of the first and third aspects of the invention;

FIG. 2 is a flow chart illustrating the patient registration process in the perioperative process;

FIG. 3 is a screen shot of a graphics tablet used in a ward during the perioperative process;

FIG. 4 is a flow chart illustrating the nurse assessment in the perioperative process;

FIG. 5 is a screen shot of a PDA used in nurse assessment during the perioperative process;

FIG. 6 is a flow chart illustrating surgeon assessment in the perioperative process;

FIG. 7 is a screen shot of a PDA used in surgeon assessment during the perioperative process;

FIG. 8 is a flow chart illustrating anaesthetist assessment in the perioperative process;

FIG. 9 is a screen shot of a PDA used in anaesthetist assessment during the perioperative process;

FIG. 10 is a screen shot of a PDA used by the surgeon during the perioperative process;

FIG. 11 is a further screen shot of a PDA used by the surgeon during the perioperative process;

FIG. 12 is a flow chart illustrating the next patient call OK process in the perioperative process;

FIG. 13 is a screen shot of a graphics tablet used in the operating theatre during the perioperative process;

FIG. 14 is a flow chart illustrating the next patient call delay process in the perioperative process;

FIG. 15 is a flow chart illustrating the patient arriving at anaesthetics room process in the perioperative process;

FIG. 16 is a flow chart illustrating the patient arriving in operating theatre process in the perioperative process;

FIG. 17 is a flow chart illustrating the patient arriving in recovery room process in the perioperative process;

FIG. 18 is a flow chart illustrating the patient returning to ward process in the perioperative process, and

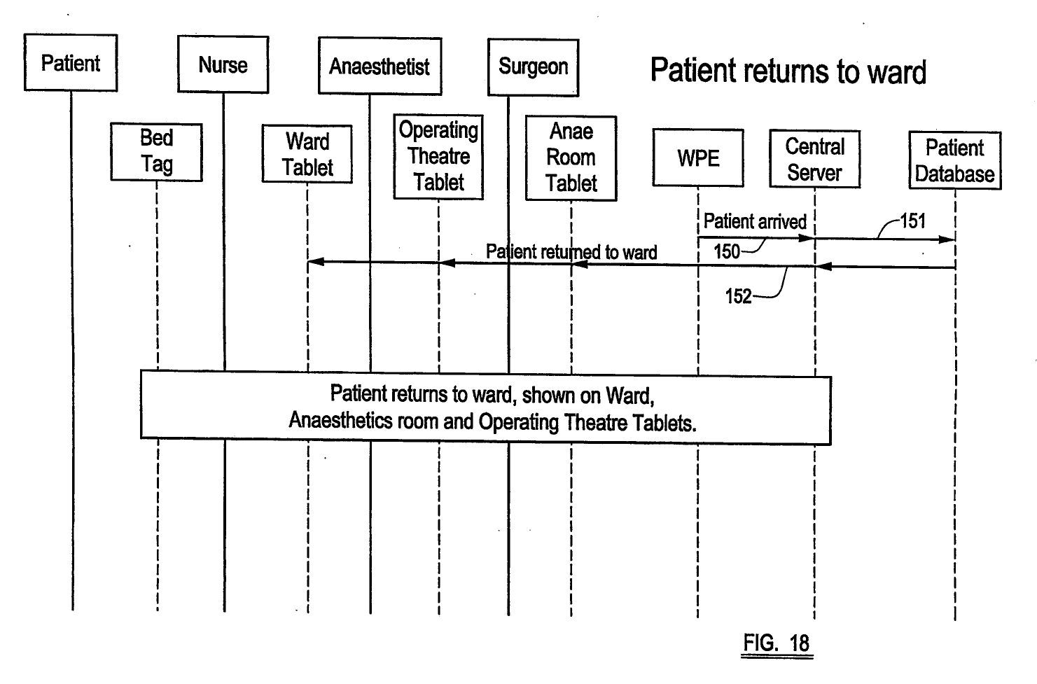

FIG. 19 is a flow chart illustrating the patient checking out process in the perioperative process.

### DETAILED DESCRIPTION OF THE PREFERRED EMBODIMENTS

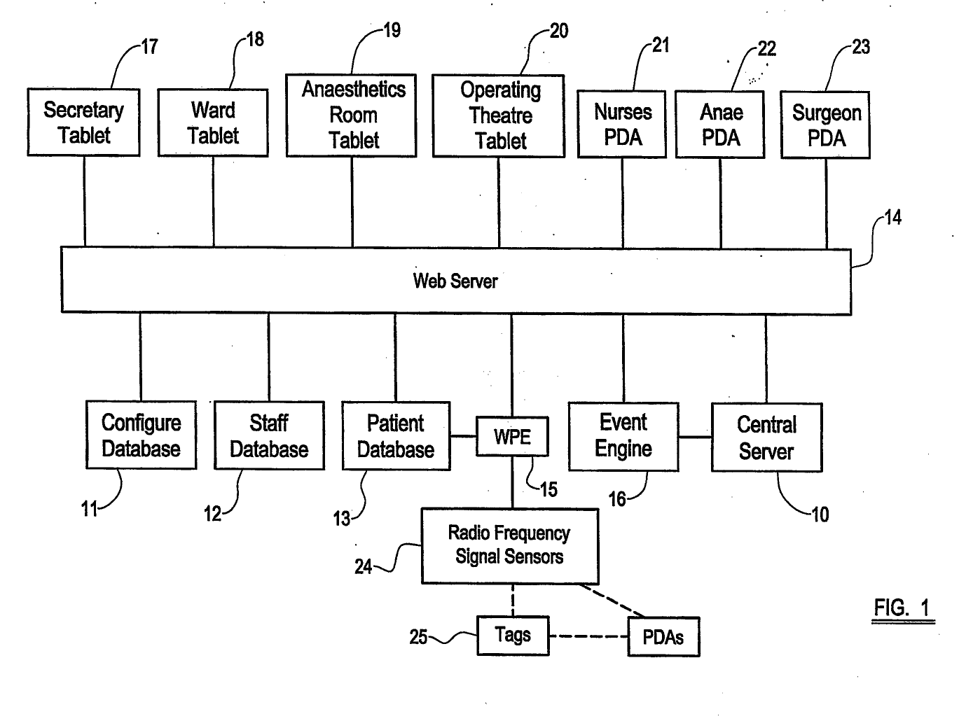

FIG. 1 is a schematic representation of a system of hardware providing a patient records system used to carry out the perioperative process of the invention. It will be appreciated that the hardware shown is representative only, and that other hardware components and configurations could be used for this process.

The system of FIG. 1 comprises a central server 10, a configuration database 11, a staff database 12 and a patient database 13, each of which is connected to a web server 14, as shown. The system further comprises a wireless positioning engine (WPE) 15, connected to the web server 14 and the patient database, as shown, and an event engine 16, connected to the web server 14 and the central server 10, as shown. The web server 14 acts as a point-of-communication between the above-mentioned components and one or more users of the perioperative process. The communication may take place via one or more input/output devices, such as computers, graphics tablets, or the like. In the present system, four such input/output devices are illustrated, a tablet 17 for use in a secretarial area, a tablet 18 for use in a ward, a tablet 19 for use in an anaesthetics room, and a tablet 20 for use in an operating theatre. It will be appreciated that further such devices could be provided. All of the above- mentioned components of the hardware are comprised in a wired network, for example an Ethernet wired local area network (LAN) or wireless area network.

The system further comprises input/output devices in the form of a number of personal digital assistant (PDAs). In the present system, three PDAs are shown, one for use by a nurse 21, one for use by an anaesthetist 22, and one for use by a surgeon 23. There are connected to the web server 14 via a wireless connection, for example an Ethernet wireless LAN. They can be used as input/output devices, for further communication between the web server 14 etc. and users of the perioperative process.

The system further comprises a network of radio frequency signal sensors 24, comprising in this example readers with antennae for reading wireless tags. These are distributed throughout the site in which the perioperative process is employed, for example a hospital. They form a wireless or wired network, which is connected to the WPE 15.

The system further comprises a number of wireless tags generally illustrated at 25. For example, a RFID tag may be attached to a patient or a bed for a patient.

In this system, the use of radio frequency signal sensors and RFID tags is described. However, it will be appreciated that other technologies could be used, such as infra red devices, Bluetooth devices or WiFi devices.

The system comprises a further input/output device, in the form of a digital camera system (not shown). The system may be integrated with one or more wireless or wired health monitoring devices (not shown).

The central server 10 and the web server 14 each comprise a computer. They each receive, process and output data to be used in the perioperative process, for example data concerning patients, staff, surgical procedures, etc. The central server comprises an embedded, intelligent microsystem, incorporating for example RBFN or MLP (radial basis function network and multilayer perceptron) technology, and controls the operation of the perioperative process, for each patient. The web server 14 is the primary interface for users of the process. It may comprise a proprietary web server interface, such as Microsoft (RTM) Internet Information Services or Apache. The interface may run customised browser-based software, using a protocol such as Hyper Text Mark-up Language (HTML), to communicate with the various input/output devices. The input/output devices may each comprise an interface running software for communication with the web server via for example HTML.

As shown in the present system, each database is stored in a separate repository, such as a memory device, which may be accessed to read data therefrom and write data thereto. Alternatively, it will be understood that one or more of the databases may be provided within the web server computer or the central server computer. The databases may be developed using an existing database software package, such as Oracle.

The staff database 12 may contain details of members of staff registered with the perioperative process, such as surgeons, anaesthetists, nurses, etc. The details concerning each member of staff which are held by the staff database may comprise the name, identification number, briefjob description, and security code, such as a password or PIN, of the member of staff.

The patient database 13 may contain patient information comprising details of one or more patients registered with the perioperative process, i. e. patients scheduled for surgery. The details concerning each patient which are held by the patient database may comprise the name, identification number, gender, age, and photograph file name of the patient, a date of admission of the patient, the ward number/name/reference code to which the patient is being admitted, the name/identification number of the members of staff (e. g. consultant surgeon, anaesthetist, nursing staff) responsible for the care of the patient, an identification number/description, date and start time of the surgery to be carried out on the patient, a type/reference code of the anaesthetic to be used on the patient, a code/identification number of the operating theatre where the surgery is to take place, known allergies or infections etc. The patient database may further comprise editable notes on each patient. It will understood that this is not an exhaustive list, and other information may be held by the patient database. The details concerning a patient may be held within a patient record, in the patient database.

The configuration database 11 contains configuration information for the perioperative process. This may comprise access control lists, detailing which members of staff have access to which parts of the system. This may comprise update configuration information, detailing how the perioperative process will update information contained in it. This may comprise validation information, detailing how the perioperative process system will validate information which it receives. This may comprise control information, detailing which information is displayed on the input/output devices. This may comprise graphical configuration information, detailing how information is presented on the input/output devices.

The event engine 16 provides real time information to the web server from the central server. The role of the event engine is to account for the present changes being made to a patient record.

The WPE 15 provides a near real time account of the movements of each RFID tag 25 (and therefore the patient or bed to which the tag is attached) throughout the hospital environment. Each RFID tag 25 is able to emit radio frequency signals. These are detected by the network of radio frequency signal sensors 24, and the detected signals are reported to the WPE 15, which uses them to determine the position of the RFID tag 25. The WPE 15 is configured and validated, and a determination of the accuracy of locating a tag 25 is made.

The WPE 15 and the network of radio frequency sensors 24 may be used to provide the location of a tag in any appropriate manner. For example, the WPE 15 may use the signals from a plurality of sensors in the network 24 to obtain accurate location information using triangulation. Alternatively, the network of radio frequency sensors 24 may comprise readers with antennae which detect a tag 25 when it comes into range of the antenna. In this case, the location information will comprise the location of the reader which detected the tag. Such a passive system will be easier to implement but less precise than a system using triangulation.

The operation of the perioperative process for each patient follows a rigorous linear flow, in order to ensure that there can be little deviation from the accepted perioperative process. The sequence of events is as follows.

A secretary uploads an operating list for a ward for the day onto the safe central server 10. This will contain the record for each patient of the ward, who is scheduled to have elective surgery that day. A record for a patient scheduled for emergency surgery can also be entered onto the list, once bed allocation status is known. The secretary uploads the operating list onto the central server using a spreadsheet. For each patient, the list contains at least the fields: date of surgery, start time of surgery, operating theatre identification, consultant surgeon and anaesthetist name, surgery order number, patient name, patient number, patient age and sex, ward identification, bed allocation (blank until arrival of patient on ward), surgery identification including side and site (free text), anaesthetic type (general or local).

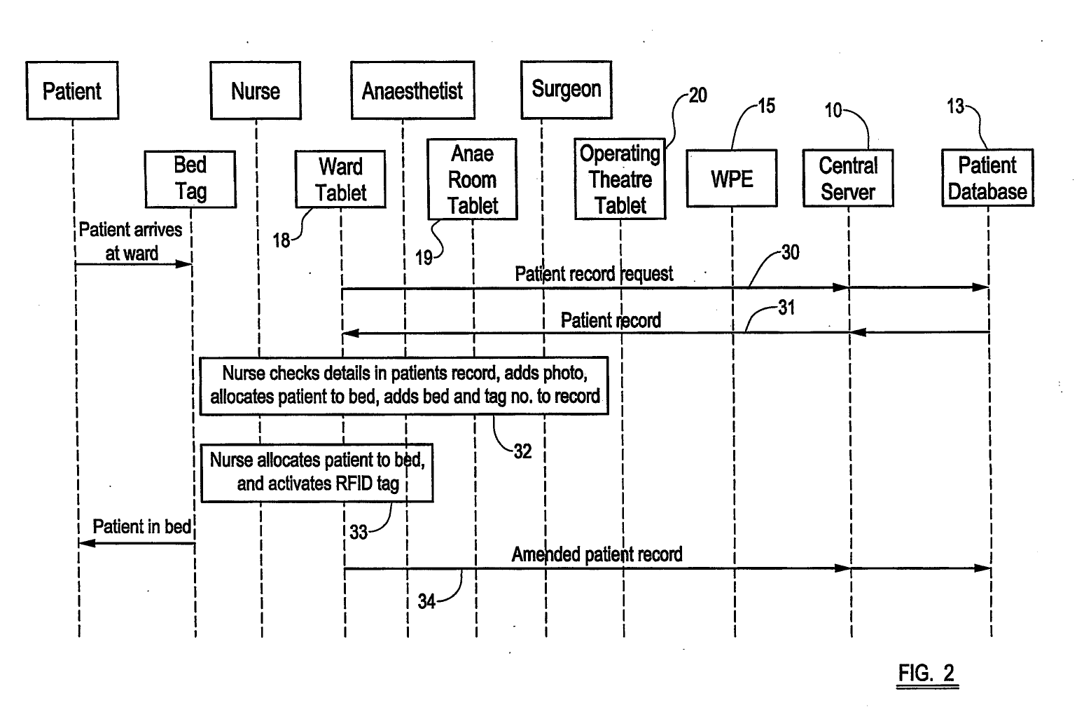

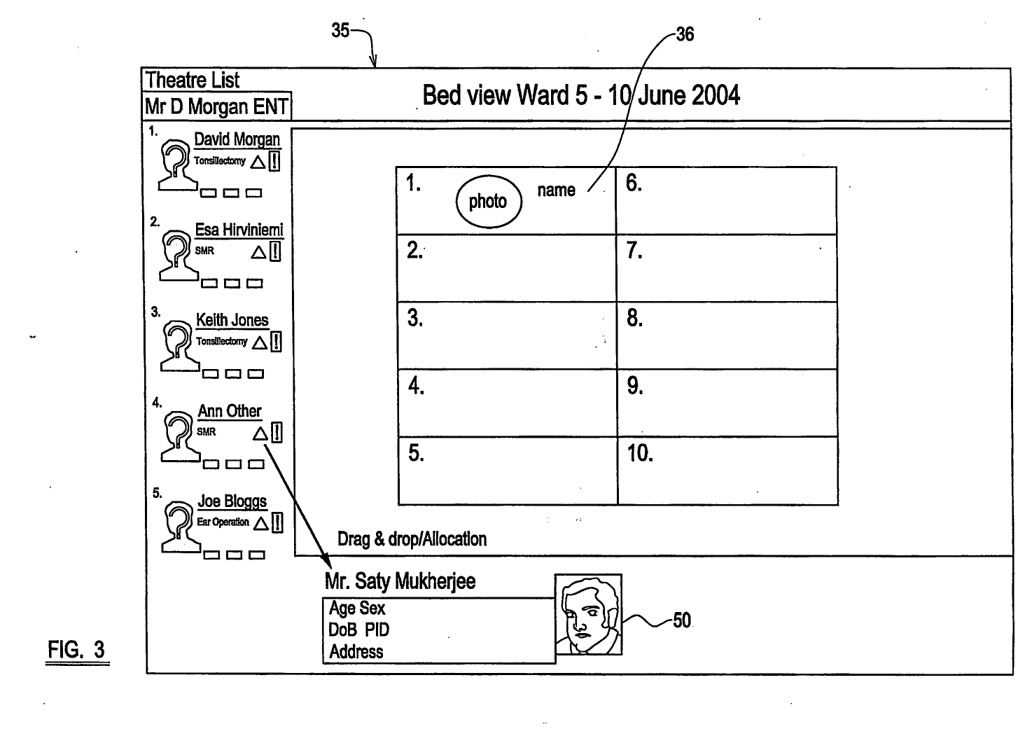

The patient arrives on the ward, and patient registration takes place. The process of patient registration is illustrated in FIG. 2. If the patient is undergoing elective surgery, a record for the patient will already exist in the patient database 13, as this will have been previously entered by secretarial staff. A nurse logs onto the perioperative process via the ward tablet 18, by entering his name and security code, e. g. a PIN or a password. This is transferred to the central server, where it is verified by comparison with the information for the nurse held in the staff database. If the nurse is registered with the perioperative process, and the entered security code matches the stored security code, the nurse is allowed access to the system. The nurse then requests the patient record from the patient database as shown at step 30, and when this is received as shown at 31, the nurse checks that the details contained in the record are correct, and makes any necessary amendments (step 32). At step 33, the patient is photographed, particularly a facial image of the patient, using the digital camera system, and the photograph file is added to the patient record (step 34). The patient is assigned to a bed, which is provided with a RFID tag, which is activated. The bed number and the RFID tag number are added to the patient record. A screen shot from the ward tablet 18 during bed allocation is shown at 35 in FIG. 3. Each bed is indicated by a status indicator 36 and is given a color code, white for bed not occupied, red for bed occupied and patient not ready for surgery, amber for bed occupied and patient partly ready for surgery (some checks performed and OK), green for bed occupied and patient ready for surgery (all checks performed and OK), and blue for bed occupied and patient returned from surgery. As the patient has just arrived on the ward, and will therefore not yet be ready for surgery, the bed assigned to the patient is color coded red, and this coding is added to the patient record. The amended patient record is then sent to the patient database.

If the patient is undergoing emergency surgery, the patient is registered as before, with the exception that the nurse creates a record for the patient, as a record will not be present in the patient database.

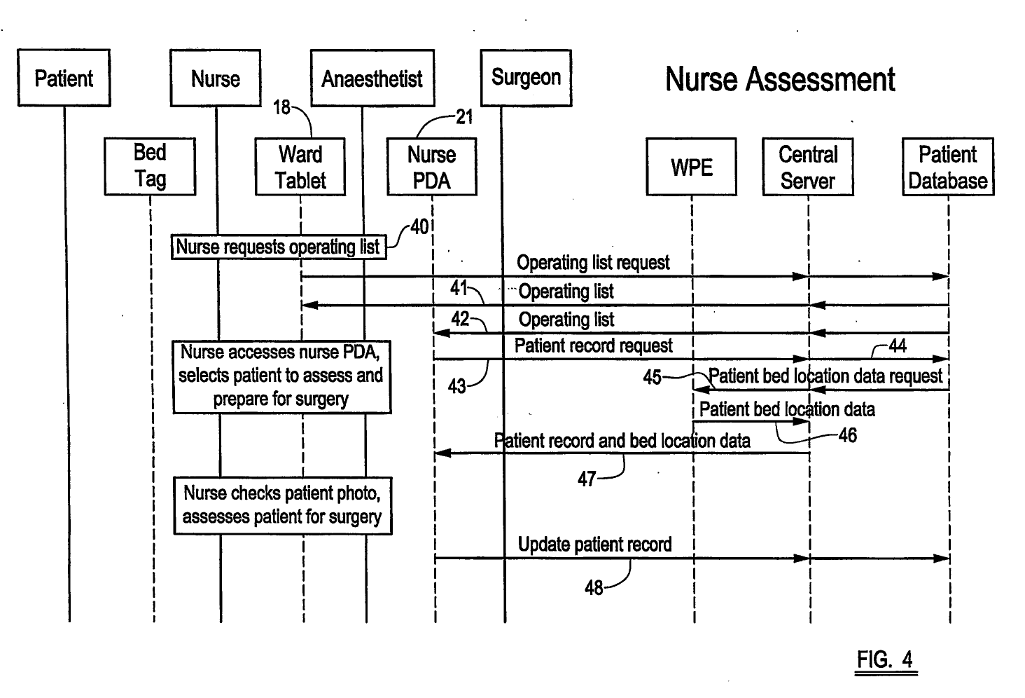

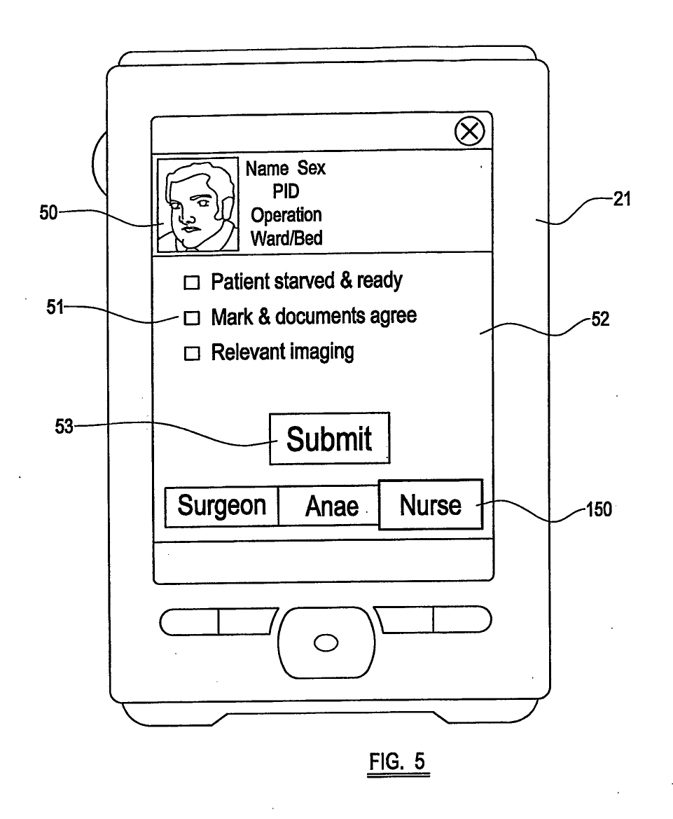

At an appropriate time, a ward nurse assesses the fitness of the patient for surgery. This process is illustrated in FIG. 4. The nurse logs onto the perioperative process using the ward tablet 18, as before, entering his name and security code. The nurse details are verified as before, and if access to the system is allowed, at step 40 the nurse then requests that the operating list for the ward for that day be sent to the ward tablet 18 or the nurse PDA 21 as shown at 41 and 42. The nurse then logs onto the nurse PDA as shown at 42, by entering their name and security code. These details are again verified, and access allowed or denied. If access is allowed, the operating list is displayed on the screen of the nurse PDA. The nurse selects the patient to be assessed and prepared for surgery, by selecting the patient from the list of patients shown in the operating list. This initiates a patient record request 43, which is sent from the nurse PDA 21 to the central server 10. The central server 10 accesses the patient record from the patient database 13 as shown at step 44. The central server 10 further requests patient bed location data from the WPE using the tag identifier from the patient information (step 45). The WPE 15 uses the network of radio frequency signal sensors 24 to determine the location of the RFID tag 25 provided on the bed assigned to the patient, and at step 46 passes this information to the central server 10. The patient record and patient bed location data are then sent to the nurse PDA 21 at step 47, and displayed on the screen of the PDA 21 as shown at 50 in FIG. 5. Alternatively on approaching the patient the WPE 15 matches the position of the PDA 21 with the patient's RFID tag 25 and initiates the appropriate patient record from the database 13 which is displayed on the PDA 21 via the server 10. The nurse then goes to the patient bed location, and first of all checks the photograph 50 of the patient shown on the nurse PDA 21 against the patient in the bed. If these match, the nurse then assesses the fitness of the patient for surgery, by carrying out various checks such as blood pressure, temperature, heart rate. The results generate verification information which is added to the patient record, via the nurse PDA 21. If the results of at least some of the checks are satisfactory, and the patient is deemed ready for surgery, the nurse indicates this by selecting the ‘patient health checks’ tick boxes on shown at 51 the screen 52 of the nurse PDA 21. The nurse then prepares the patient for surgery. This verification information is then sent to the central server 10 of the system as shown at 48, by the nurse selecting a ‘submit’ box 53 on the screen of the nurse PDA 21, as illustrated in FIG. 5. The central server 10 uses the information to update the patient record held in the patient database 13.

The event engine 16 is informed of the change to the patient record, and notifies the surgeon, via the surgeon PDA 23.

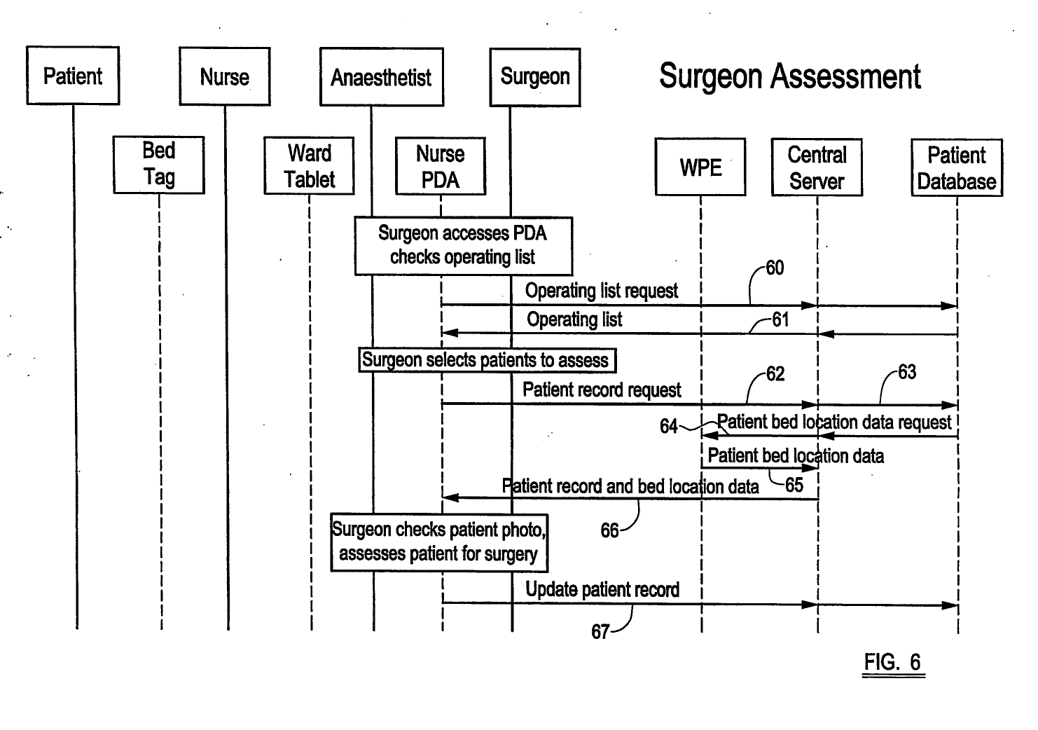

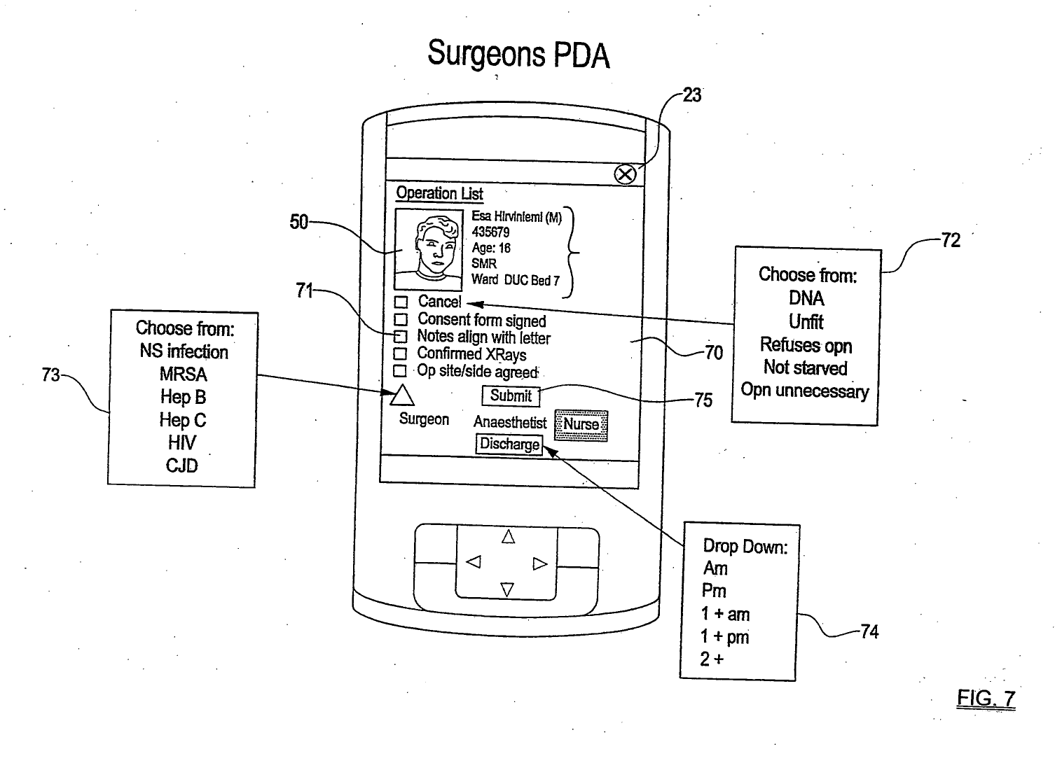

The surgeon then assesses the fitness of the patient for surgery. This process is illustrated in FIG. 6 and the surgeon PDA 23 is shown in FIG. 7. The surgeon accesses the surgeon PDA 23 and logs onto the perioperative process by entering his name and security code. The surgeon details are verified as before, and if access to the system is allowed, the surgeon then requests that the operating list 100 for that day be sent to the surgeon PDA 22 as shown at 60, and the operating list 100 is returned as shown at 61 and is displayed on the screen 70 of the surgeon PDA 23. The surgeon selects the patient to be assessed for surgery, by selecting the patient from the list of patients shown in the operating list. This initiates a patient record request 62, which is sent from the surgeon PDA 23 to the central server 10. The central server 10 accesses the patient record from the patient database 13 as shown at 63. The central server further requests patient bed location data from the WPE 15 as shown at 64, using the tag identifier from the patient information. The WPE 15 uses the network of radio frequency signal sensors 24 to determine the location of the RFID tag provided on the bed assigned to the patient, and passes this information to the central server 10 as shown at 65. At step 66, the patient record and patient bed location data are then sent to the surgeon PDA 23, and displayed on the screen 70 of the PDA 23 as shown in FIG. 7. The surgeon arrives on ward, and goes to the patient bed location, and first of all checks the photograph 50 of the patient shown on the surgeon PDA 23 against the patient in the bed. If these match, the surgeon then assesses the fitness of the patient for surgery, by carrying out various checks. These include checking the patient consent letter has been signed, checking the patient notes, checking any previous diagnostic results e. g. X-rays, agreeing the operation side and site with the patient. The screen 70 of the surgeon PDA 23 is provided with a tick box for each of these checks, shown at 71. If the result of a check is satisfactory, the surgeon indicates this by selecting the appropriate tick box on the surgeon PDA screen 70. If the results of at least some of the checks are satisfactory, the verification information is sent to the server 10 and, if appropriate, the patient status information is updated: for example, the patient is deemed ready for surgery, and the color code of the bed assigned to the patient changed from red to amber or green, as appropriate. The screen of the surgeon PDA also provides a ‘cancel’ tick box. If the surgeon deems that the surgery should be cancelled, this tick box is selected, and the reason for cancellation (surgery not necessary or inappropriate or patient did not arrive) chosen from a drop down list of options 72. Other information can also be entered, such as a patient infection (in this example selected from a drop down list 73) or discharge time (list 74). A patient allergy or other information could also be entered. The information input into the surgeon PDA 23 is then sent to the central server 10 of the system shown at 67, by the surgeon selecting a ‘submit’ box 75 on the screen 70 of the surgeon PDA 23. The central server uses the transmitted verification information to update the patient record held in the patient database.

The event engine is again informed of the change to the patient record, and notifies the anaesthetist, via the anaesthetist PDA 22.

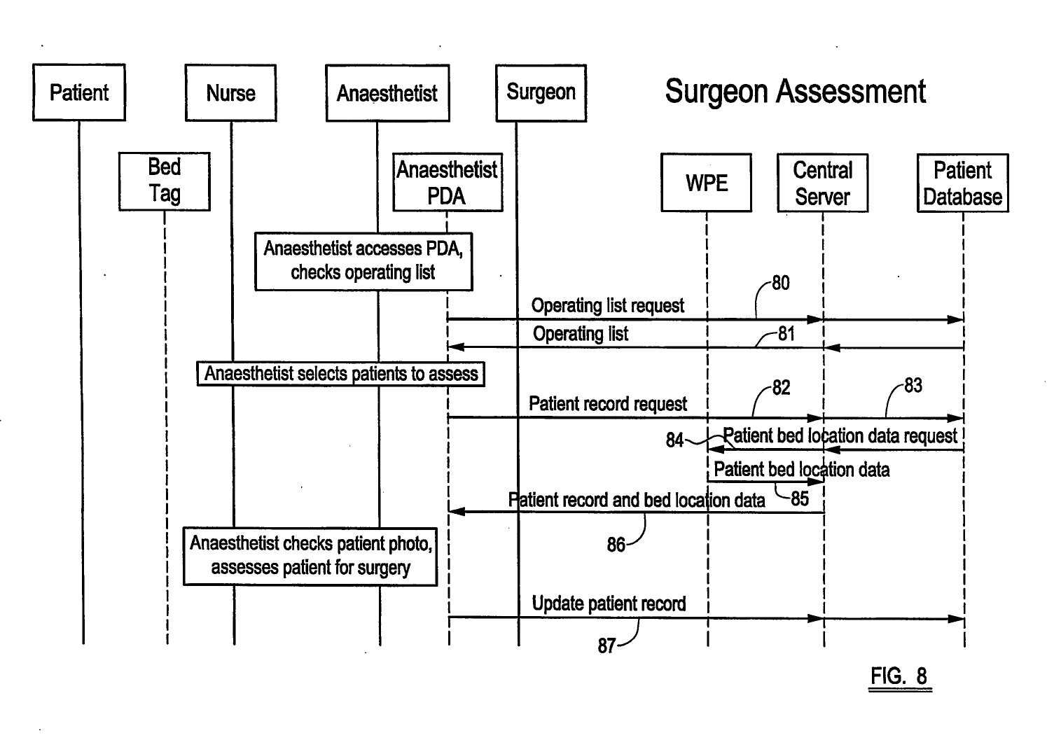

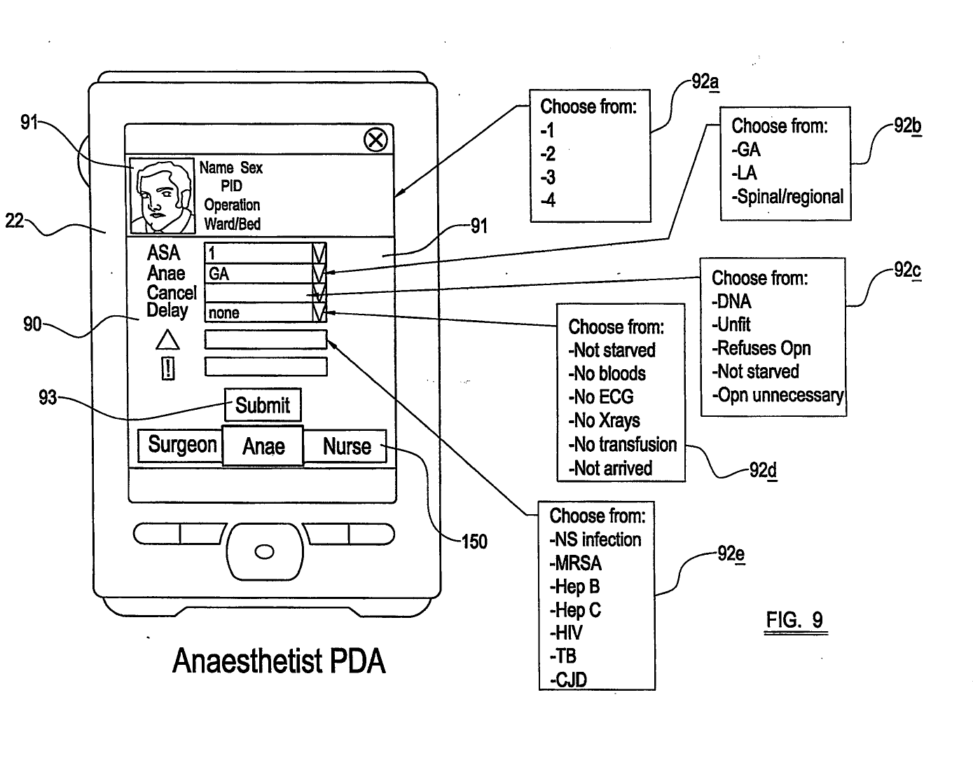

The anaesthetist then assesses the fitness of the patient for surgery. This process is illustrated in FIG. 8 and the anaesthetist PDA shown in FIG. 9. The anaesthetist accesses the anaesthetist PDA 22 and logs onto the perioperative process by entering his name and security code. The anaesthetist details are verified as before, and if access to the system is allowed, the anaesthetist then requests as shown at step 80 that the operating list for that day be sent to the anaesthetist PDA, and this is returned at step 81 and displayed on the screen 90 of the anaesthetist PDA 22. The anaesthetist selects the patient to be assessed for surgery, by selecting the patient from the list of patients shown in the operating list. This initiates a patient record request 82, which is sent from the anaesthetist PDA 22 to the central server 10. The central server accesses the patient record from the patient database 13 as shown at 83. The central server 10 further requests patient bed location data from the WPE 15 as shown at 84. The WPE 15 uses the network of radio frequency signal sensors 24 to determine the location of the RFID tag 25 provided on the bed assigned to the patient, and passes this information 85 to the central server 10. The patient record and patient bed location data are then sent to the anaesthetist PDA as shown at 86, and displayed on the screen 90 of the PDA 22. The anaesthetist arrives on ward, and goes to the patient bed location, and first of all checks the photograph 91 of the patient shown on the PDA 22 against the patient in the bed. If these match, the anaesthetist then assesses the fitness of the patient for surgery, by carrying out various checks. These include checking that the patient has been starved, checking patient blood test results, checking patient ECG results, checking any previous diagnostic results e. g. X-rays, checking patient blood transfusion. The screen of the anaesthetist PDA is provided with tick boxes 91, which may each have a drop down list of options 92a, 92b, 92c, 92d, 92e. The anaesthetist selects from the lists of options, for example from options 1,2, 3, or 4 in the ‘ASA’ box 92a, from the option of a general anaesthetic (GA) or local anaesthetic (LA) in the ‘Anae’ box 92b, and from the options of proceed or reason for cancelling (not starved, no bloods, no ECG, no X-rays, no transfusion, not arrived, not fit) in the ‘cancel’ box 92c and the record, for example, did not arrive (DNA), refuses operation, not stored, operation unnecessary. The fourth box 92d of the screen of the anaesthetist PDA indicates whether or not the surgery is to be delayed and the reason for delay include not stored, no blood tests, no ECG, no X-rays, no transfusion, did not arrive (DNA). Box 92e allows the anaesthetist to enter patient infection information. The verification information input into the anaesthetist PDA 22 is then sent to the central server of the system as shown at 87, by the anaesthetist selecting a ‘submit’ box 93 on the screen 90 of the anaesthetist PDA 22. The central server 10 uses the information to update the patient record held in the patient database 13.

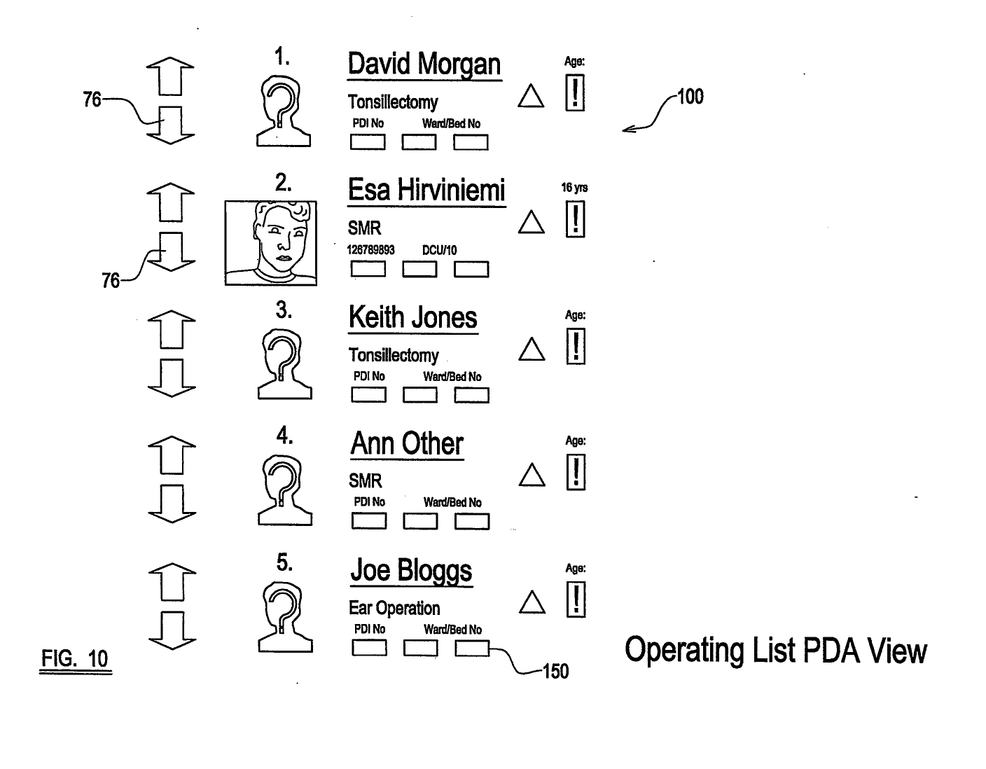

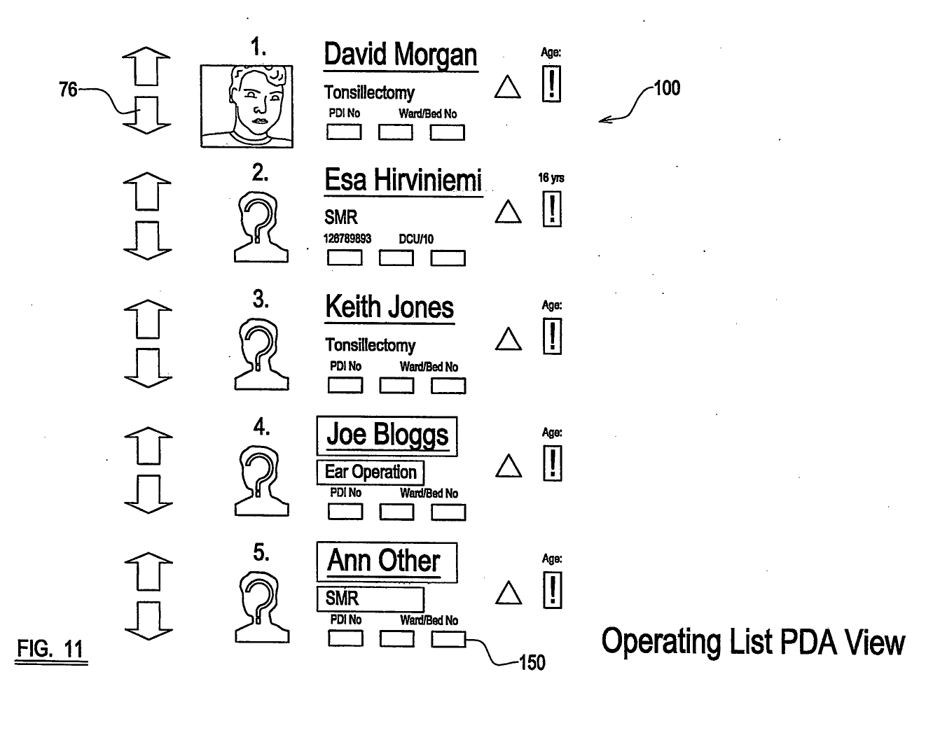

The surgeon may check the operating list 100 using the surgeon PDA 23 (or the tablet in the operating theatre 20). The surgeon may re-order the operating list via the surgeon PDA 23, using up and down arrows, as illustrated in FIGS. 10 and 11. The option to add an emergency surgery to the list is also provided. Any changes made to the operating list 100 are sent to the central server 100, and the modified list is accessible by the anaesthetist and the ward.

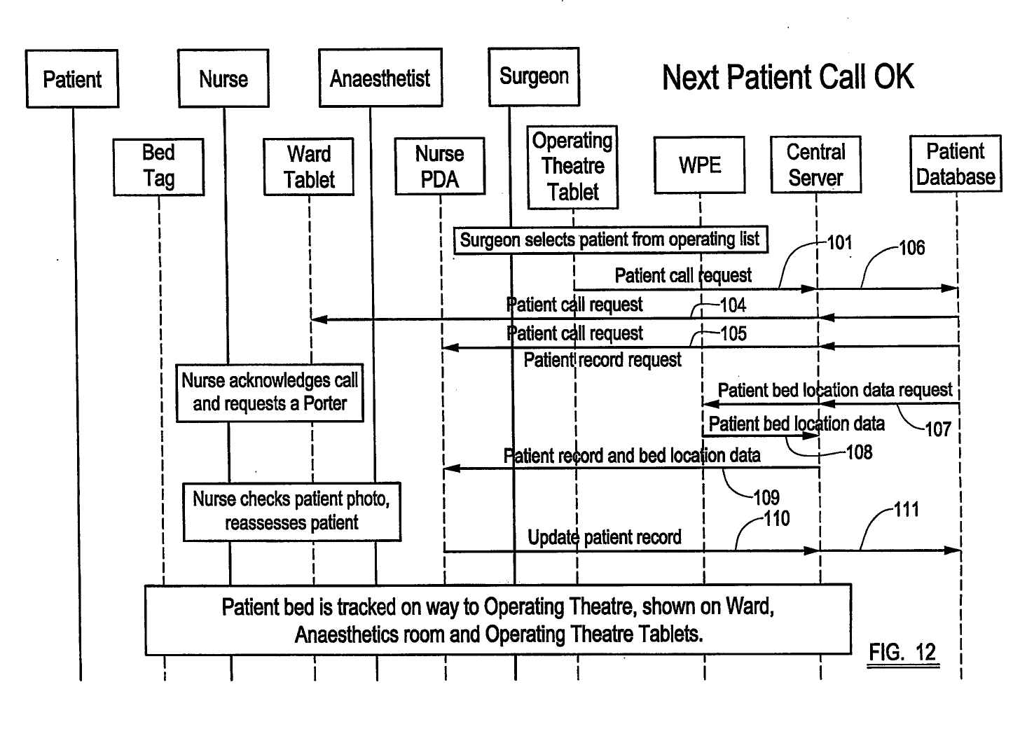

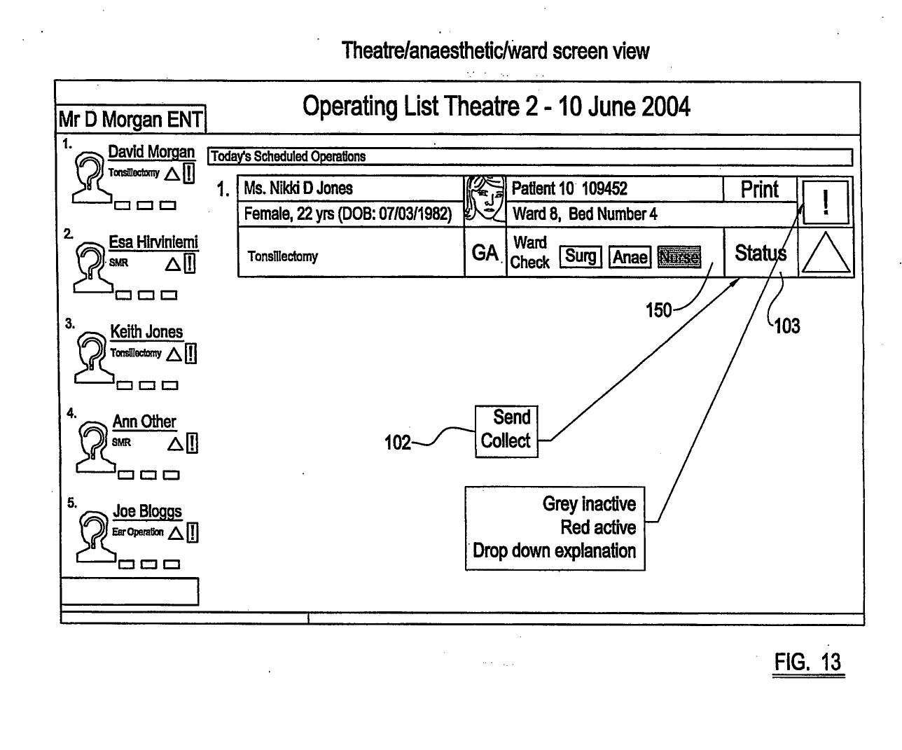

When the surgeon is ready for a patient from the operating list, he initiates a call for the patient, as illustrated at 101 in FIG. 12. The surgeon accesses the perioperative process using the tablet 20 in the operating theatre, and calls up the operating list 100. The surgeon selects the patient, and chooses ‘send’ from the drop down options 102 of the ‘status’ box 103 on the tablet (see FIG. 13). The patient call request 101 is sent to the central server 10, which sends the request to the ward tablet 18 and the nurse PDA 21 as shown at 104, 105 respectively. The patient call request is acknowledged, and a porter is requested. The central server 10 accesses the patient database 13 as shown at 100, and retrieves the patient record as shown at 107. The central server 10 further requests patient bed location data from the WPE 15 as shown at 108 using the tag identifier from the patient information. The WPE 15 uses the network of radio frequency signal sensors 24 to determine the location of the RFID tag 25 provided on the bed assigned to the patient, and passes this information to the central server 10. As shown at 109, the patient record and patient bed location data are then sent to the nurse PDA 21, and displayed on the screen of the PDA 21. The nurse then goes to the patient bed location, and first of all checks the photograph 50 of the patient shown on the nurse PDA 21 against the patient in the bed. If these match, the nurse then reassesses the fitness of the patient for surgery, by carrying out various checks such as blood pressure, temperature, heart rate patient starved, mark and documents agree and relevant imaging is present. The results of these are added to the patient record, via the nurse PDA 21. If the results of at least some of the checks are still satisfactory, the patient is deemed still ready for surgery, and the nurse indicates this by sending this information to the central server 10 of the system as shown at 110. The central server 10 uses the verification information to update the patient record held in the patient database 13 as shown at 111. The nurse instructs the porter to take the patient (in their bed) to the operating theatre. When the patient bed leaves the ward, the movement of the RFID tag provided on the patient/bed is noted by the network of radio frequency signal sensors 24. This is communicated to the WPE 15, which informs the central server 10 that the patient is being moved, which in turn alerts the surgeon and anaesthetist that the patient is on the way, using the tablets 18,19 in the operating theatre and the anaesthetics room, and/or the respective PDA 22,23.

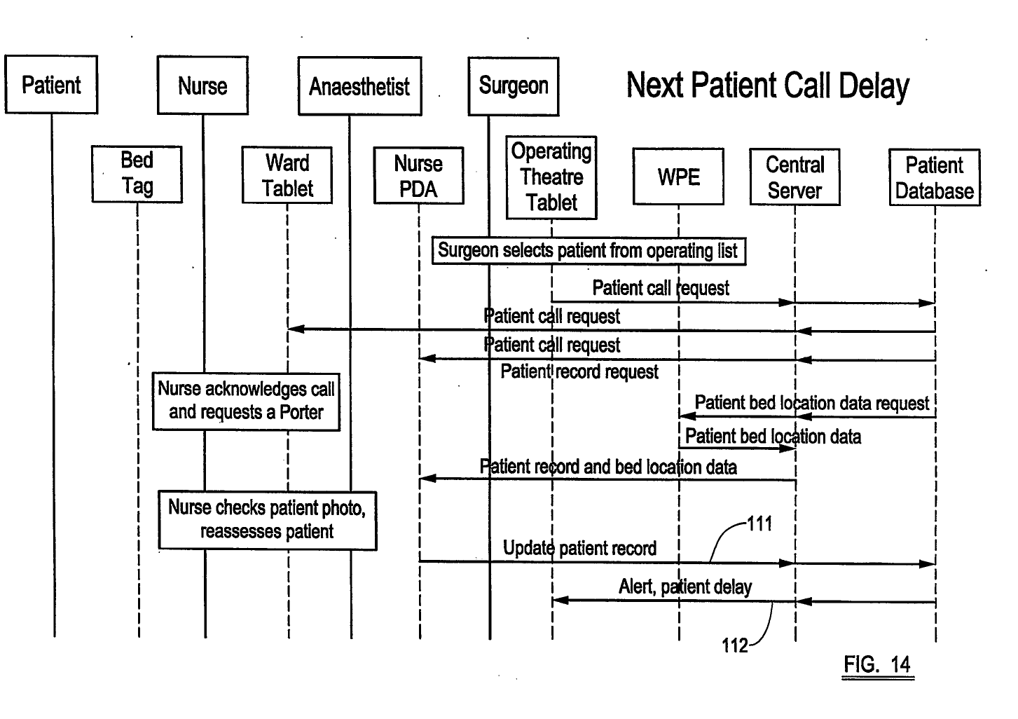

If the results of at least some of the checks carried out by the nurse are not now satisfactory, the patient is deemed not to be ready for surgery, and the nurse indicates this by sending this information at step 111 to the central server 10 of the system. The central server 10 alerts the surgeon and anaesthetist that this is the case by sending a notification to the appropriate input/output devices, here, using the tablets 18,19 in the operating theatre and the anaesthetics room. This is illustrated at step 112 in FIG. 14.

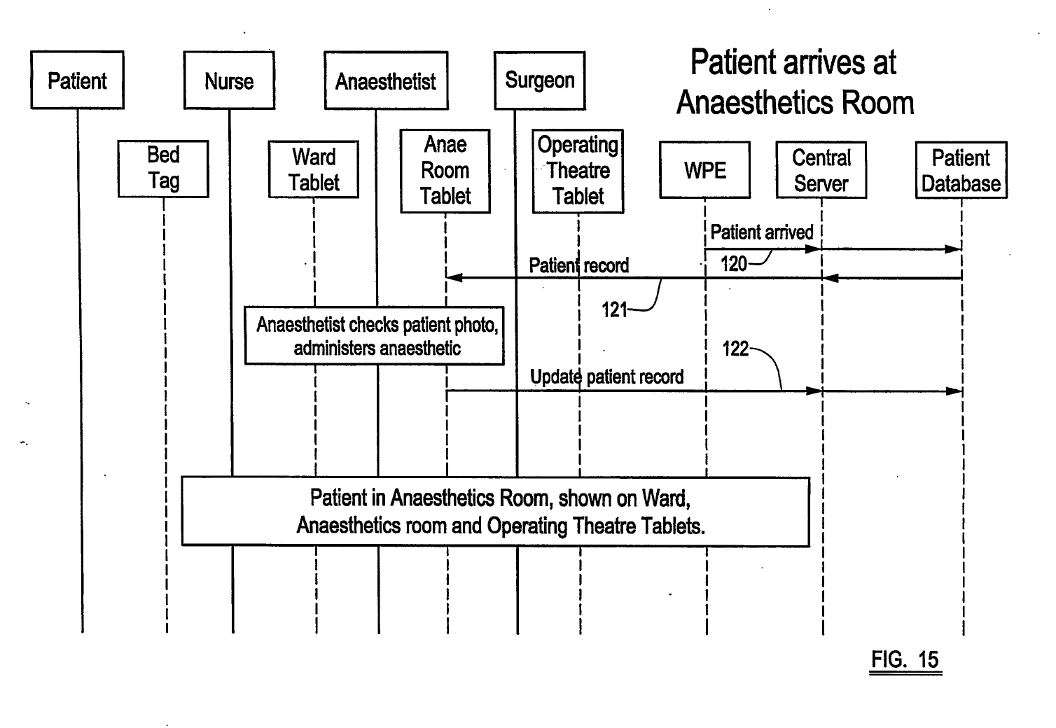

When the patient arrives in the anaesthetics room, the RFID tag 25 provided on the patient bed is detected by radio frequency signal sensors in the room. The WPE 15 deduces that the location of the patient bed is the anaesthetics room, and passes this information to the central server 10 as shown at 120 in FIG. 15. The server 10 verifies that the patient which is in the anaesthetics room is the patient which has been called, by comparing the identification of the tag which is located in the anaesthetics room with the identification of the tag which is provided on the patient/bed of the patient which has been called, which information is accessed from the patient database. The server 10 may alternatively compare the patient details on the operating list 100 with the patient details of the patient record comprising the tag identifier of the detected tag 25. If the patient who has arrived in the anaesthetics room is not the patient who has been called, an alert is generated by the central server, which is displayed on the tablet 19 of the anaesthetics room. If the patient who has arrived in the anaesthetics room is the patient who has been called, the central server 10 notifies the anaesthetics room tablet 19, the operating theatre tablet 20 and the ward tablet 18 that this is the case, and sends the patient record to the tablet 19 in the anaesthetics room as shown at 121. The anaesthetist checks the photograph of the patient against the patient in the anaesthetics room. If these coincide, the anaesthetist proceeds to administer the anaesthetic to the patient, and adds details of the dosage and estimated recovery time to the patient record, which is sent to the central server 10 as shown at 122 and stored in the patient database 13. This process is illustrated in FIG. 15.

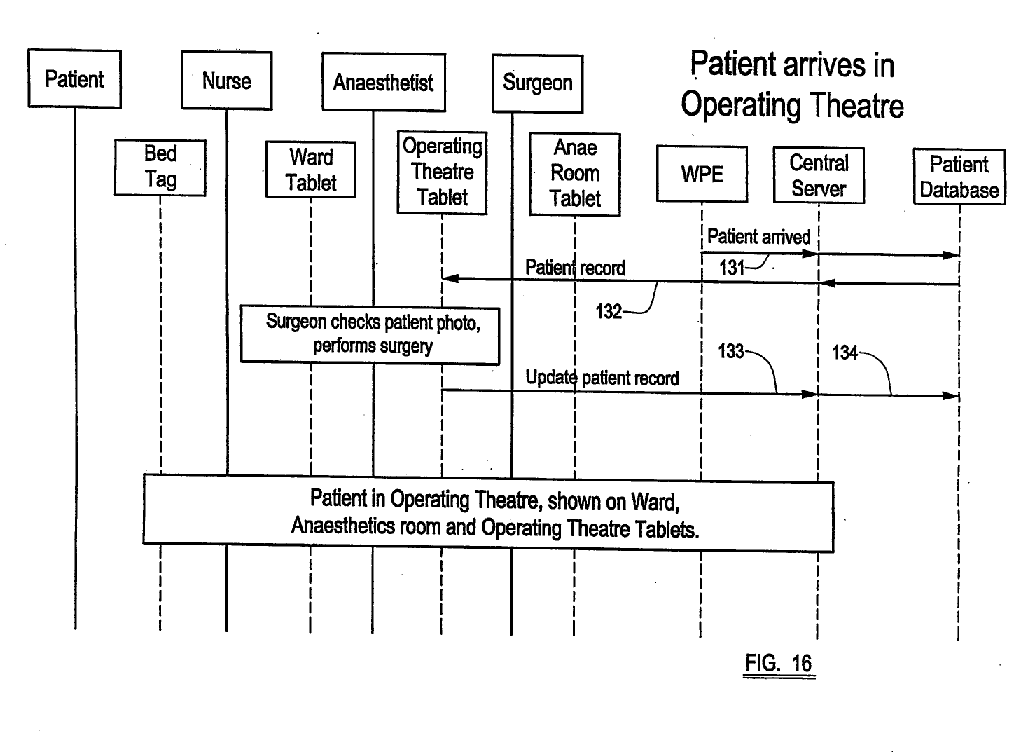

The patient is then moved to the operating theatre. When the patient arrives in the operating theatre as shown in FIG. 16, the RFID tag 25 provided on the patient bed is detected by radio frequency signal sensors in the theatre. The WPE 15 deduces that the location of the patient bed is the operating theatre, and passes this information to the central server 10 as shown at 131. The central server notifies the anaesthetics room tablet 19, the operating theatre tablet 20 and the ward tablet 18 that the patient has arrived in the operating theatre, and retrieves the patient information and sends to the tablet 20 in the operating theatre as shown at 132. The surgeon checks the photograph of the patient against the patient in the operating theatre. If these coincide, the surgeon, anaesthetist and scrub nurse agree the type of operation, and the operation site and side, using information from the patient record displayed on the tablet in the operating theatre, to avoid WSS. The surgeon then proceeds with the operation, and, when completed, adds a summary of the surgery, details of any problems, and medication advice to the patient record, which is sent to the central server 10 and stored in the patient database as shown at 134. If a biopsy/test is necessary a PDA with a printer can print off the appropriate patient's details onto a label as the WPE synchronises the RFID tag with the PDA and sends the patient's details from the database via the server, thus mitigating against mislabelling of specimens.

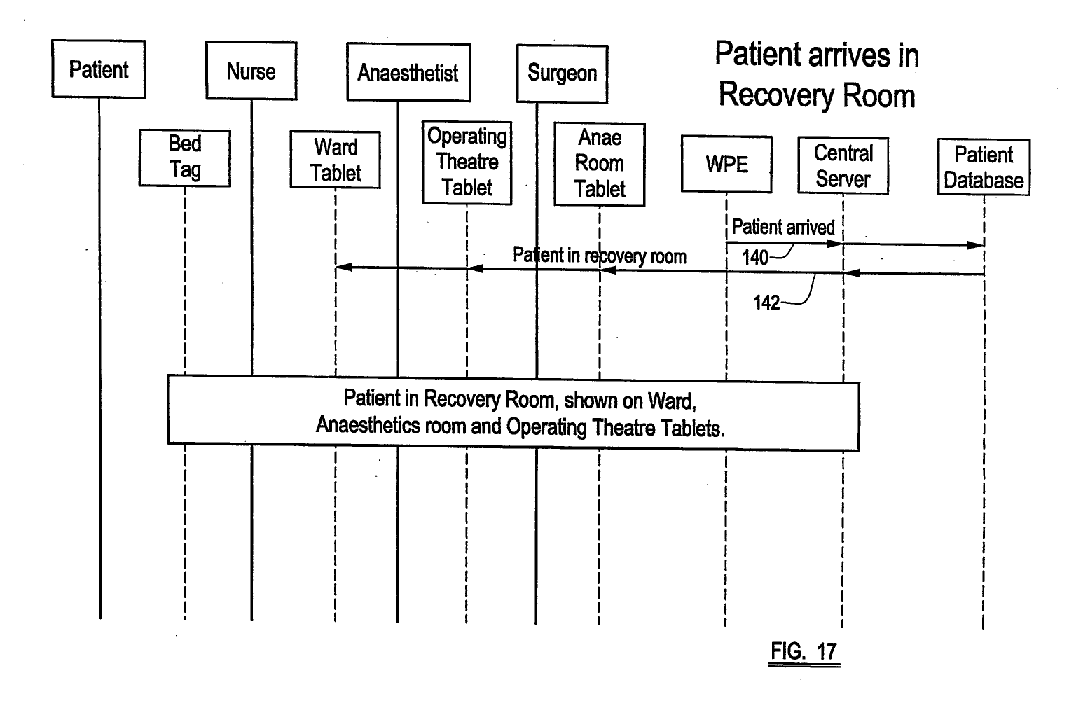

The patient is then taken to the recovery room. When the patient arrives in the recovery room, the RFID tag 25 provided on the patient bed is detected by radio frequency signal sensors 24 in the room. The WPE 15 deduces that the location of the patient bed is the recovery room, and passes this information to the central server 10 as shown at 140 in FIG. 17. The central server 10 updates the patient database 13 as shown at 141 and notifies the anaesthetics room tablet 19, the operating theatre tablet 20 and the ward tablet 18 that the patient is in the recovery room as shown at 142. When the patient is fully recovered the nurse in the recovery room sends a message to the ward nurse's tablet 18 and PDA 21 requesting collection of the patient whose bed position and photograph 50 is displayed on the ward nurse's PDA 21/tablet 18.

When appropriate, the patient is taken back to the ward. When the patient arrives in the ward, the RFID tag 25 provided on the patient bed is detected by radio frequency signal sensors 24 in the ward. The WPE 15 deduces that the location of the patient bed is the ward, and passes this information to the central server 10 as shown at 150. The server 10 verifies that the patient has been taken to the correct ward by accessing the patient database 13 as shown at 151. The central server notifies the anaesthetics room tablet 19, the operating theatre tablet 20 and the ward tablet 18 that the patient has arrived in the ward as shown at 152.

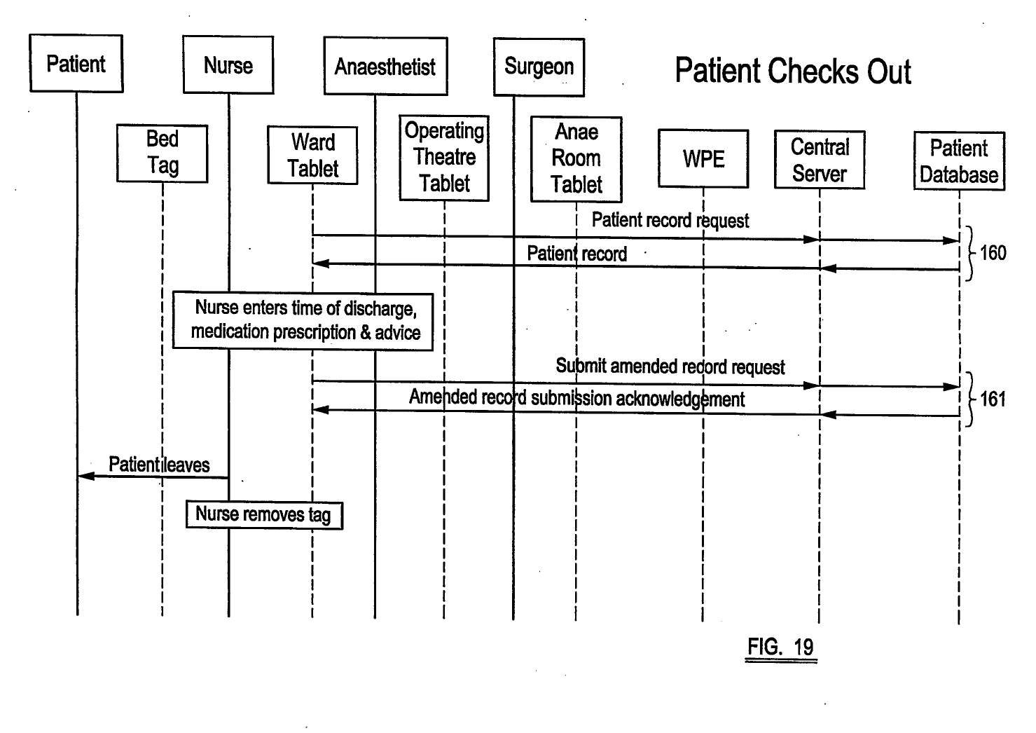

When the patient has sufficiently recovered, he is discharged from the hospital. The nurse uses the tablet 18 in the ward to access the patient record as shown at 160, and adds the time of discharge and any medication advice to this. The amended patient record is then stored in the patient database, and this is acknowledged as shown at 161. The nurse then removes the RFID tag from the bed of the patient. This process is illustrated in FIG. 19.

On each of the screens of the PDA's 21, 22, 23 and tablets 17,18, 19, 20 a status bar shows a box corresponding to the nurse, anaesthetist and surgeon, and the color of each box is set in accordance with status information showing whether the check to be performed by that person have been carried out, for example as shown in the verification information.

For example red may indicate no check has been done, amber that some checks have been passed but not all, and green that all checks have been done and passed. The status bar 150 thus provides a simple indication of the patient readiness for surgery at a glance.

The verification information generally refers to information indicating that one or more checks have been performed and that the patient is ready for a next stage of the process. As can be seen from the above, there is little room for deviation from this medical process, in this example the perioperative process. The only deviation that is permitted is when the patient fails required checks. Under these circumstances, the patient is moved back one stage, until the identified problems are rectified. Verification of the identity of the patient is carried out at numerous occasions, by comparison with a photograph, and using RFID tags. Thus the likelihood of carrying out surgery on the wrong patient is small. The surgery to be performed and the site of the surgery, is again checked at several occasions, thus reducing the possibility of WSS.

The above process can equally be applied to any patient process in hospital requiring identification such as but not exclusively endoscopy, radiology, pathology test, physiology test, biochemical test, haematology test. Although the description herein shows a perioperative process, it will be apparent that the invention may be applied to any medical process as appropriate, such as admission for investigation, drug administration or review by medical staff (eg a ward round).

It will be apparent that other variations on the system described herein may be envisaged. For example, the input/output devices may communicate directly with the wireless tags. The purpose of such communication might be as simple as retrieving the tag identifier for forwarding to the server to obtain patient information from the patient record. Alternatively, it might be envisaged that the tag holds information that may be retrieved by the input/output device, such as a copy of the patient record or a part thereof. A tag might be associated with a monitor or other piece of equipment, such as an ECG, and the input/output device could retrieve data held by the tag.

The system described herein will also have administrative advantages, in that it will allow measurement of the efficiency of resource allocation and use within the hospital, as well as organization of operations.

When used in this specification and claims, the terms “comprises” and “comprising” and variations thereof mean that the specified features, steps or integers are included. The terms are not to be interpreted to exclude the presence of other features, steps or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

I claim:

1. A patient record system operable to store a patient record corresponding to a patient and comprising patient information, the patient record system being operable during a medical process to
receive a request for patient information from an input/output device,
retrieve patient information from the patient record,
transmit the patient information to the input/output device,
receive verification information from the input/output device, and
identify the patient as being ready for a next stage of the medical process in accordance with the verification information.

2. The patient record system of claim 1, wherein the patient record includes a tag identifier corresponding to a wireless tag associated with the patient, the patient record system being operable in response to the request for patient information from an input/output device to
obtain location information of the wireless tag corresponding to the tag identifier, and
transmit the location information to the input/output device.

3. The patient record system of claim 2 operable to transmit the location information with the patient information.

4. The patient record system of claim 1 operable to
receive location information of a wireless tag having a tag identifier indicating the presence of the wireless tag at a specific location,
identify the patient record including the tag identifier of the wireless tag, and
verify the patient is expected at that location.

5. The patient record system of claim 4, wherein the patient record system is operable to generate an alarm if the location is incorrect.

6. The patient record system of claim 2, wherein the patient record system is operable to supply patient information from the patient record to an input/output device at the location if the location is correct.

7. The patient record system of claim 3, wherein the input/output device comprises an input/output device associated with the location.

8. The patient record system of claim 1, wherein the medical process comprises a perioperative process.

9. The patient record system of claim 1, wherein the input/output device comprises a portable device such as a personal digital assistant.

10. The patient record system of claim 1, wherein the step of receiving verification information comprises
generating a checklist,
receiving an input in response to the check list, and
generating verification information in accordance with the input.

11. The patient record system of claim 1, wherein the patient information comprises status information and wherein the status information is modified in response to the verification information.

12. The patient record system of claim 2, wherein the location information is displayed as a plan of a building with the patient location indicated on the plan.

13. The patient record system of claim 1, wherein the patient information comprises status information and wherein at least a portion of a display on the input/output device is color coded in accordance with the status information.

14. The patient record system of claim 1, wherein the patient information comprises an image of the patient and transmitting the patient information includes transmitting the image.

15. The patient record of claim 1, wherein receiving the request for patient information comprises receiving a list of patients with an indication corresponding to one of the list of patients.

16. The patient record system of claim 1 operable, on receipt of the verification information, to send a notification to another input/output device.

17. A medical process for a patient, comprising
creating a computerized record of patient information including an image of the patient, and,
at least once during the perioperative process, accessing the computerized record of patient information and checking that the patient image matches the patient.

18. The medical process of claim 17, wherein the computerized record of patient information includes one or more of: patient name, identification number, gender, age, date of admission; ward number/name/reference code; staff name/identification number; identification number/description, date and start time of surgery; surgery site; type/reference code of the anaesthetic; code/identification number of operating theatre where the surgery is to take place; and editable notes.

19. The medical process of claim 17, wherein the computerized record of patient information includes details of surgery site on the patient, and the record is used to check the details of the surgery site prior to surgery.

20. The patient record system of claim 1, further comprising a server, wherein the input/output device communicates with the server to create the computerized record of patient information, and wherein the input/output device communicates with the server to access the computerized record of patient information.

21. The patient record system of claim 20, wherein the input/output devices may comprise any of a computer, a graphics tablet, a PDA.

22. A method of supplying patient information comprising;
storing a patient record comprising patient information,
receiving a request for patient information from an input/output device,
retrieving patient information from the patient record,
transmitting the patient information to the input/output device,
receiving verification information from the input/output device, and
identifying the patient as being ready for a next stage of a medical process in accordance with the verification information.

23. The method according to claim 22 comprising associating a wireless tag having a tag identifier with the patient information and,
storing the tag identifier in the patient record, and in response to the request,
obtaining location information of the wireless tag corresponding to the tag identifier, and
transmitting the location information to the input/output device.

24. The method according to claim 23 comprising determining a position of the wireless tag, receiving the tag identifier, accessing the patient record containing the tag identifier, and displaying the patient information on an input/output device.

25. The method of claim 23, wherein the computerized record of patient information is displayed on the input/output device which is located in proximity to the position of the wireless tag.

26. The method according to claim 24, wherein the display is used to check that the patient undergoing the medical process matches the patient record.

27. The method of claim 26, wherein the display comprises an image of the patient.

28. The method of claim 23, wherein the wireless tag comprises a RFID tag.

29. The method of claim 26, determining location information using one or more radio frequency signal sensors and a wireless positioning engine.

30. The method of claim 22, further comprising the step of printing a label containing patient information.

31. The method of claim 30, wherein the label is a specimen label.

32. A system for implementing the method of claim 22, comprising
a tag,
a tag position detector,
a server, and
one or more than one input/output device that communicates with the server for creating, accessing, and displaying the patient record.
